(19) United States
(12) Patent Application Publication
(10) Pub. No.: US 2008/0089957 A1
(43) Pub. Date: Apr. 17, 2008

(54) Monocotyledon plant indications extract compositions, method of preparation and pharmaceutical compositions containing them

(76) Inventors: Shui-Tein Chen, Taipei (TW); Jung-Feng Hsieh, Taipei (TW)

Correspondence Address: Andrews & Kurth, L.L.P., Suite 1100, 1350 I Street N.W., Washington, DC, 20005, US

(21) Appl. No.: 11/545,442

(22) Filed: Oct. 11, 2006

(51) Int. Cl. A61K 36/899; A61K 36/88

(52) U.S. Cl. 424/725; 424/750

(57) ABSTRACT

The present invention relates to a method for immuno-modulation in an organism by using a protein extract from a monocotyledon plant. The present invention also relates to a method for inhibiting nitrite production or anti-oxidation activity in an organism by using an organic extract from a monocotyledon plant. The present invention also relates to a method for regulating uric acid in an organism by using 6-aminopurine analogues.

### BACKGROUND OF THE INVENTION

1. Field of Invention

The present invention relates to the new indications and compositions of monocotyledon plant extracts, methods to prepare them, their use as cosmetics and medicaments and the pharmaceutical compositions containing them.

2. Description of the Related Art

Many monocotyledon plants provide high value crops such as rice, wheat, sorghum, millet and herbage. Rice is a semi aquatic crop that benefits from flooded soil conditions during part or all of the growing season. In many countries, rice is a staple food. However, in addition to using the grains from rice, The biological activity of rice straw extract has not been reported.

Wheat is grown worldwide, is the most widely used cereal and is available in five main wheat market classes. They include the four common wheat (Triticum aestivum L.) classes: hard red winter, hard red spring, soft red winter and white. The fifth class is durum (Triticum turgidum L.). Common wheats are used in a variety of food products such as bread, cookies, cakes, crackers and noodles. In general, the hard wheat classes are milled into flour used for bread, and the soft wheat classes are milled into flour used for pastries and crackers. In addition to using the grain from wheat plants, people want to extract other bioactive molecules from whole wheat plants for further use, such as extracting bioactive molecules from young leaves, but still do not have a viable method to obtain and maintain the bioactive molecules from wheat plants.

Therefore, a need still exists in the related art to provide new compositions of monocotyledon plant extracts, a method to prepare them, their use as cosmetics and medicaments and the pharmaceutical compositions containing them to migrate the foregoing problems.

### SUMMARY OF THE INVENTION

An objective of the present invention is to provide new compositions of monocotyledon plant extracts with its biological activities.

Another aspect of the present invention is to provide an isolated compound of having formula I:

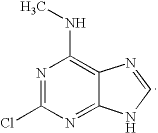

Another aspect of the present invention is to provide an organic solvent extract obtained from a monocotyledon plant

Another aspect of the present invention is to provide a water-soluble extract obtained from a monocotyledon plant.

Another aspect of the present invention is to provide a method for immuno-modulation in an organism.

Another aspect of the present invention is to provide a method for inhibiting nitrite production in an organism.

Another aspect of the present invention is to provide a method for anti-oxidation activity in an organism.

Another aspect of the present invention is to provide a method for regulating uric acid in an organism.

Other aspects, advantages and novel features of the invention will become more apparent from the following detailed description when taken in conjunction with the accompanying drawings.

### BRIEF DESCRIPTION OF THE DRAWINGS

FIG. 1 is a graph showing cells treated with lipopolysaccharides (LPS), water-soluble wheat extract, acetonitrile wheat extract and acetone wheat extract on nitrite production,

FIG. 2 is a graph showing cell viability percentage of cells treated with LPS, water-soluble wheat extract, acetonitrile wheat extract and acetone wheat extract,

FIG. 3 is a graph showing DPPH-scavenging activity of water-soluble wheat extract, acetonitrile wheat extract and acetone wheat extract,

FIG. 4 is a graph showing inhibition of xanthine oxidase by different fractions of water-soluble wheat extracts,

FIG. 5 is a graph showing inhibitory effects on XOD activity by 2-cholro-6(methylamino) purine, allopurinol, 6-aminopurine and 4-aminopyrazolo[3,4-d]purimidine,

FIG. 6 is a graph showing tyrosinase inhibitory activity assay for the wheat organic solvent extracts,

FIG. 7 is a graph of further purification of wheat tyrosinase inhibitor,

FIG. 8 is a graph of further purification of wheat phosphodiesterases inhibitor, and

FIG. 9 is a scheme of purification steps of rice extract.

### DETAILED DESCRIPTION OF THE INVENTION

A monocotyledon plant according to the present invention refers to a monocotyledon plant that is used in agriculture. For example, the plant is cultivated by humans for the purpose of nourishment or for technical purposes, particularly industrial purposes. Thus the monocotyledon plant in accordance with the present invention may be rice, rye, barley, oats, wheat, millet, rice, maize or herbage.

In one preferred embodiment of the present invention, the monocotyledon plants may be wheat, rice, barley, oats, rye, maize or herbage. Monocotyledon plants being wheat, rice and herbage plants are preferred, and monocotyledon plants being wheat and rice are particularly preferred.

The monocotyledon plant extracts show several functions such as the following. 1) Bioactive monocotyledon plant protein extracts, such as wheat grass proteins and rice grass proteins, that may show immuno-modulation activity and increase the expression of CD16⁺CD56⁺ markers on NK cells. The protein extracts obtained from monocotyledon plants preferably are greater than 30 kDa. 2) Bioactive monocotyledon plant organic solvent extracts that show inhibition of nitrite production and anti-oxidation activity. The organic solvent monocotyledon plant extract may be extracted by ACN and acetone and are crude organic solvent extracts. 3) Bioactive water-soluble monocotyledon plant extracts show regulation of uric acid. The water-soluble monocotyledon plant extracts are crude extracts, and may contain 6-aminopurine-based analogues including allopurinol, 2-chloro-6(methylamino)purine, 6-aminopurine and 4-aminopyrazolo[3,4-d]pyrimidine allopurinol. The formula of 6-aminopurine-base analogues include

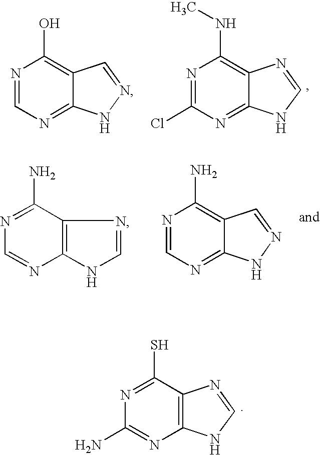

### The molecular formula of 6-aminopurine-base analogues were C₅H₄N₄O, C₆H₆ClN₅, C₅H₅N₅, C₅H₅N₅ and C₅H₅N₅S, respectively.

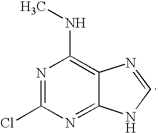

is currently preferred. 4) Bioactive water-soluble monocotyledon plant extracts show regulation of blood glucose levels, tyrosinase activity and phosphodiesterases activity. The water-soluble monocotyledon plant extracts are crude extracts.

Because the monocotyledon plant extracts show the above merits, the monocotyledon plant extracts may be applied for pharmaceutical compositions and cosmetic compositions.

In one preferred embodiment of the present invention, the overall functions of monocotyledon plant extracts are summarized in the following Table 1.

TABLE 1
The overall functions of monocotyledon plant extracts are
summarized.

| Research | | |
|---|---|---|
| Direction | Mechanism | Function |
| Metabolism | Bioactive of | Anti-oxidation |
| | monocotyledon plant | |
| | protein | |
| Metabolism | SOD | Anti-oxidation |
| Immune | NK Cell Stimulation | increase the populations of |
| modulaton | and proliferation | immune cells |
| Immune | Inhibition effect on | Anti-inflammation |
| modulaton | nitrite production | |
| Metabolism | DPPH | Anti-oxidation |
| Metabolism | Xanthine oxidase | Regulation of the uric acid |
| | inhibitor | |
| Metabolism | Insulin | Regulation of the blood glucose |
| | | level |
| Metabolism | Tyrosinase inhibitor | Regulation of the tyrosinase |
| | | activity |
| Metabolism | Phosphodiesterases | Regulation of the |
| | inhibitor | phosphodiesterases activity |

To facilitate an understanding of the present invention, a number of terms and phrases as used herein are defined below:

“Plant material” is understood in general to mean whole fresh plants, whole dried plants, parts of fresh plants or parts of dried plants. Parts of plants, for example, may be plant leaves, stalks, or stems.

“Plant extract” is understood in general to mean both plants and parts of plants, for example and preferably dried or dehydrated and ground, or also extracts of such plants or parts of plants obtained using at least one aqueous and/or organic solvent and being present in a standard liquid or in particular solid form used in pharmacy, cosmetics or dietetics.

“Immuno-modulation” is understood in general to mean activity associated with the immune system, immunity, induced sensitivity, and allergy.

“Organism” as used herein refers to a non-human animal, including, without limitation, farm animals such as cattle, sheep, pigs, goats and horses; domestic mammals such as dogs and cats; laboratory animals including ferrets, hares and rabbits, rodents, such as mice, rats, hamsters, gerbils, and guinea pigs; non-human primates, including chimpanzees. The term “animal” may also include, without limitation; birds, including domestic, wild and game birds such as chickens, turkeys and other gallinaceous birds, ducks, geese, and the like, as well as amphibians, fish, insects, reptiles, etc. The term does not denote a particular age. Thus, adult, embryonic, fetal, and newborn individuals are intended to be covered.

“Effective amount” as used herein refers to that amount of the extract which will contribute to immuno-modulation ability of the composition.

Unless defined otherwise, all technical and scientific terms used herein have the same meaning as commonly understood by people of ordinary skill in the art to which this invention belongs. Also, all publications, patent applications, patents and other references mentioned herein are incorporated by reference.

The present invention relates to a protein extract obtained form monocotyledon plant by following steps:

providing monocotyledon plant materials,

treating the monocotyledon plant materials with ammonium sulfate, wherein the concentrations of ammonium sulfate are about 30% to about 100% (w/v), and

obtaining the protein extract form ammonium sulfate.

Preferably, the protein extracts are respectively collected from 40% (w/v), 40% to 60% (w/v), 60% to 70% (w/v), 70% to 80% (w/v) and 80% to 100% (w/v) fractions.

Preferably, the molecular weight of the protein extract is greater than 30 kDa.

The present invention relates to a xanthine oxidase inhibitor having formula I:

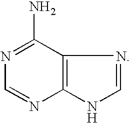

### The molecular formula was C₅H₅N₅.

The present invention also relates to a water extract obtained form monocotyledon plant by following steps:

(1) dissolving the monocotyledon plant material in water and heated at about 85° C. for about 10 minutes,

(2) collecting water extract and a first pellet from the dissolving monocotyledon plant material,

(3) re-dissolving water extract in water, and

(4) subjecting the re-dissolving water extract to a column chromatography on a C18 packed column with eluting H₂O:AcCN:H₃PO₄ (87:13:0.01) and H₂O:AcCN:H₃PO₄ (70:30:0.01) to obtain active components.

Preferably, elution started with H₂O:AcCN:H₃PO₄ (87:13:0.01) and is conducted for about 30 minutes at a flow rate of about 12 ml/min, is changed to H₂O:AcCN:H₃PO₄ (70:30:0.01) for about 15 minutes, and then is changed back to H₂O:AcCN:H₃PO₄ (87:13:0.01), the fractions are respectively collected.

More preferably, the fractions are collected at elution times of 0 to 4, 4 to 6.5, 6.5 to 10, 10 to 19, 19 to 23, 23 to 41 and 41 to 59 minutes.

The present invention also relates to a water extract obtained form monocotyledon plant by following steps:

(1) dissolving the monocotyledon plant material in water and heated at about 85° C. for about 10 minutes,

(2) collecting water extract and a first pellet from the dissolving monocotyledon plant material,

(3) re-dissolving water extract in water, and

(4) subjecting the re-dissolving water extract to a C18 open column using elution with H₂O:AcCN (100:0) and H₂O:AcCN (0:100).

Preferably, a first fraction collected at fraction time of 6.5 to 10, the fraction is further subjected to a C18 packed column eluting with H₂O:AcCN:TFA (100:0:0.01) and H₂O:AcCN:TFA (0:100:0.01) and a second fraction is collected for further purified.

Preferably, the second fraction is subjected to a C18 open column and a C18 packed column.

The present invention also relates to an organic extract obtained form monocotyledon plant by following steps:

(1) dissolving the monocotyledon plant material in water and heated at about 85° C. for about 10 minutes,

(2) collecting water extract and a first pellet from the dissolving monocotyledon plant material, and

(3) treated the first pellet with acetonitrile for 6 hours to obtain an acetonitrile extract and a second pellet.

Preferably, the second pellet is further re-dissolving with acetone for 6 hours to obtain an acetone extract and a third pellet.

Preferably, the third pellet is further treated with hexane to obtain a hexane extract.

The present invention also relates to a method for immuno-modulation in an organism comprising administering to the organism an effective amount of a composition comprising a protein extract of a monocotyledon.

The present invention also relates to a method for inhibiting nitrite production in an organism, comprising administering to the organism an effective amount of the composition comprising the acetonitrile extract.

The present invention also relates to a method for anti-oxidation activity in an organism, comprising administering to the organism an effective amount of the composition comprising the acetonitrile extract.

The present invention also relates to a method for anti-oxidation activity in an organism, comprising administering to the organism an effective amount of the composition comprising the acetone extract.

The present invention also relates to a method for regulating uric acid in an organism, comprising administering to the organism an effective amount of the composition comprising the isolated compound of having formula I:

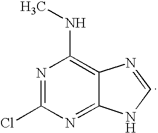

The examples illustrate the following advantages of the monocotyledon plants extract composition, the method of preparation, the pharmaceutical composition and the cosmetic compositions containing the monocotyledon plants extract.

The following non-limiting examples are intended to provide additional understanding of the invention.

### EXAMPLES

The following examples illustrate various aspects of the present invention but do not limit the claims in any manner whatsoever.

### Example 1

### Method for Preparing Wheat Protein Extract

1.1 Plant Materials

Wheat grass (Triticum aestivum) was grown individually in 30 cm diameter×15 cm high containers. Water and fertilizer were applied by drip irrigation. The plants were grown in chambers in a day (25° C.) and night (18° C.) temperature cycle and a 16 hour and 8 hour day and night photoperiod for 8 to 10 days. After harvesting, the wheat grass was milled in a laboratory-scale milling machine to obtain wheat grass juice. Then the wheat grass juice was filtered through a filter paper (Whatmam No. 1) and a 0.22 μm filter membrane to obtain a filtrate. Then the filtrate was quickly wrapped in an aluminum foil pouch and the pouch was immediately submerged in liquid nitrogen to minimize proteolytic activity. Wheat filtrate samples were stored at −80° C. before use.

1.2 Protein Precipitation

1.2.1 Methods

Wheat filtrate samples were salted with solid ammonium sulfate with 0 to 100% (w/v), respectively. The salting steps are well known in the art and suitable methods would be apparent to a skilled person in the art. Treated samples were collected respectively at 40, 40˜60, 60˜70, 70˜80 and 80˜100% saturation of ammonium sulfate. Each fraction was collected by centrifugation under 12,000 g for 40 min at 4° C. and dissolved in a phosphate buffer (50 mM; pH 7.5). The dissolved fraction was dialyzed by a dialysis membrane (SnakeSkinT Dialysis Tubing, 10K MWCO, Pierce biotechnology, Inc., 35 FT/PKG) extensively against the phosphate buffer at 4° C. for 24 hours. The dialyzed protein was then concentrated by lyophilization followed by further purification steps. The purified protein was suspended (1 g/5 mL) in chilled (−20° C.) 10% trichloroacetic acid (TCA) in acetone containing 0.07% β-mercaptoethanol (β-ME). The mixture was incubated at −20° C. for 4 hours then centrifuged at 12,000 g for 40 minutes to obtain a pellet. The pellet was rinsed three times (5 mL) with chilled (−20° C.) acetone containing 0.07% β-ME and centrifuged at 12,000 g for 40 minutes between rinses. The fluid was removed and the pellet was dried slowly under a nitrogen blanket.

1.2.2 Results

Table 3 shows the results of fractional precipitation of proteins from wheat grass (Triticum aestivum) using ammonium sulfate. Wheat filtrate samples were salted with solid ammonium sulfate with 0 to 100% (w/v) saturation to obtain wheat protein extracts. A total of five fractions were obtained, and the total yield of these fractions was 95.89%. Among all the obtained fractions, the highest protein content (56.69%) was present in the fraction precipitated with 40 to 60% saturation of ammonium sulfate.

TABLE 2
Fractional precipitation of proteins from wheat grass (Triticum
aestivum) using ammonium sulfate

| Ammonium | Volume | Total protein | Protein content | Yield |
|---|---|---|---|---|
| sulfate (%) | (mL) | (mg) | (%) | (%) |
| Crude extracts | 410 | 866.6 | 100.00 | |
| 0–40 | 47 | 88.65 | 10.22 | 10.22 |
| 40–60 | 65 | 491.35 | 56.69 | 66.92 |
| 60–70 | 58 | 163.2 | 18.83 | 85.75 |
| 70–80 | 49 | 53.64 | 6.18 | 91.94 |
| 80–100 | 54 | 34.16 | 3.94 | 95.89 |

1.3 Protein Quantification and Analysis

1.3.1 Method for Protein Quantification and Analysis

Wheat protein samples collected at 40, 40 to 60, 60 to 70, 70 to 80 and 80 to 100% saturation of ammonium sulfate were analyzed by a modified Bradford protein quantification assay. The modified Bradford protein quantification assay was utilized to overcome interference of the 8 M urea and 60 mM DTT present in the solubilization solution. To perform the modified Bradford protein quantification assay for quantifying the collected proteins are well known in the art and suitable methods would be apparent to a skilled person in the art, so further description of the assay process is not provided.

Wheat protein extracts collected at 40, 40 to 60, 60 to 70, 70 to 80 and 80 to 100% saturation of ammonium sulfate were also analyzed by SDS-PAGE. To perform and analyze by SDS-PAGE are well known in the art and suitable methods would be apparent to a skilled person in the art, so further description of the SDS-PAGE process is not provided.

Wheat protein extracts collected at 40, 40 to 60, 60 to 70, 70 to 80 and 80 to 100% saturation of ammonium sulfate were analyzed by two-dimension (2D) gel electrophoresis. The two-dimension gel electrophoresis was used to analysis the protein fractions precipitated from wheat filtrate samples. Spots of interest were cut from 2D-gels for further In-gel digestion. The cut spots were sliced into mm³ pieces and then washed three times with 200 μl water and 50 mM ammonium bicarbonate buffer (pH 8.0) in 50% acetonitrile for 15 minutes. Gel pieces obtained from in-gel digestion were further analyzed by MALDI-TOF mass spectrometry to determine peptide mass spectra and to obtain peptide mass fingerprint data.

The peptide mass fingerprint data was further analyzed by a Swiss-Prot Peptide mass mapping, a particularly successful method for the identification of proteins, to identify proteins. Protein selection criteria are: a good match of at least five fragments from a single 2-D gel spot against a single protein sequence entry in the database, the high coverage value and the human-origin sequence. Proteins meeting these criteria were considered as candidates.

Finally, proteins were classified by their functions. For functional classification, a BGSSJ program (http://bgssj.sourceforge.net/) was used and the program was developed by the present inventors. The BGSSJ program is an XML-based Java application that organizes lists of interesting genes or proteins for biological interpretation in the context of Gene Ontology that organizes information by molecular function, biological processes and cellular components for a number of different organisms.

1.3.2 Results

The fractional purified proteins collected at 40, 40˜60, 60˜70, 70˜80 and 80˜100% saturation of ammonium sulfate were classified into three groups: molecular function, cellular components and biological process. The precipitation proteins were found to include more than 120 kinds including, ribulose-1,5-bisphosphate carboxylase/oxygenase, Cu/Zn superoxide dismutase (SOD), phosphoribulokinase, putative hypersensitive-induced reaction protein, fructose-bisphosphate aldolase, reversibly glycosylated polypeptide, ribulose bisphosphate carboxylase, nucleoside diphosphate kinase, cyclophilin-like protein, 2-cys peroxiredoxin BAS1, alpha 2 subunit of 20S proteasome, ADP-glucose pyrophosphorylase small subunit, fructose-1,6-bisphosphatase, heat shock proteins, phosphoglycerate mutase, beta-amylase, isoprene synthase, ribulose bisphosphate carboxylase, ferredoxin-NADP(H) oxidoreductase, glutathione transferase, malate dehydrogenase, putative malate dehydrogenase, alpha-L-arabinofuranosidase/beta-D-xylosidase isoenzyme ARA-I, hypothetical protein, peroxidases, triose-phosphate isomerase precursor, ascorbate peroxidase, ribulose-5-phosphate-3-epimerase, dehydroascorbate reductase, putative 3-beta hydroxysteroid dehydrogenase/isomerase, putative glyoxalase, hypothetical protein, cytosolic 3-phosphoglycerate kinase, UTP-glucose-1-phosphate uridylyltransferase, phosphoglycerate kinase, ribulose-bisphosphate carboxylase, alcohol dehydrogenase I, dehydroascorbate reductase, ascorbate peroxidase and putative lactase.

### Example 2

### Immuno-Modulation Activity of Wheat Protein Extracts

2.1 Plant Materials

Wheat grass (Triticum aestivum) was grown individually in 30 cm diameter×15 cm high containers. Water and fertilizer were applied by drip irrigation. The plants were grown in chambers with a 24° C. and 18° C. day-night temperature cycle and a 16 hour and 8 hour day-night photoperiod for 8 to 10 days. After harvesting, the wheat grass was milled in a laboratory-scale milling machine to obtain wheat grass juice. Then the wheat grass juice was filtered firstly through a 0.22 μm filter membrane and subsequently a centrifugal filter device (Centricon cut-off: 30 kDa, Amicon Millipore Co. U.S.A) to obtain a filtrate. The filtrate (molecular weight<100 kDa and >100 kDa) was then lyophilized and stored at −80° C. until use.

2.2 Isolation of Umbilical Cord Blood (UCB) Mononuclear Cells

Human UCB from six healthy volunteers was drawn into EDTA-coated tubes. The blood was collected right after a full-term baby was delivered and before the placenta separated from the uterus. Using aseptic procedures, an 18-gauge needle was inserted into the umbilical vein and umbilical cord blood was drawn for tests. Samples were stored at room temperature and processed within 24 hours after collection. The umbilical cord blood (100 mL) was processed using density gradient centrifugation with an equal volume of Biocoll separating solution without breaking (density 1.077; AUTOGENBIOCLEAR). Centrifuging was performed for 30 minutes at 1,900 rpm (300 g) at room temperature in a swinging-bucket rotor, and then a mononuclear cell layer in the interphase was collected. The buffy coat interface was retrieved, and the cells were washed twice with Dulbecco's phosphate buffered saline ([PBS] pH7.5; SIGMA) and centrifuged for 5 min at 1,500 rpm at room temperature. The cells were re-suspended in a complete culture medium (consisting of 90% RPMI-1640, 2 mM L-glutamine, 4.5 g/L glucose 10 mM HEPES, 1.5 g/L sodium bicarbonate, 1 mM sodium pyruvate, 100 units/mL penicillin, 100 μg/mL streptomycin, 0.25 μg/mL amphotericin), and the culture medium was then supplemented with 10% fetal bovine serum (FBS). Mononuclear cells isolated through these procedures were prepared at a final concentration of 10⁶ cells/mL.

2.3 Wheat Protein Extracts Treatment of UCB Mononuclear Cells

The UCB mononuclear cells isolated from the six umbilical cord blood specimens were placed in culture flasks at 10⁶ cells/mL density in preparation for the wheat protein extract treatment. After seeding of cells, wheat protein extracts (molecular weight>30 kDa and molecular weight<30 kDa, 100 μg/mL) were respectively added to each culture for 7 days at 37° C. To conduct flow cytometry, cells (1-2×10⁶) were pelleted and re-suspended in 3 mL of PBS. A PBS buffer (100 μL) containing 10 μL of fluorescence-conjugated antibody was added to the cell suspension for labeling. After incubation at 4° C. for 40 minutes, all samples were then centrifuged at 1,500 rpm for 5 minutes, followed by washing of the pellets twice with PBS. The suspension was removed, and 0.2 ml of cold PBS at 4° C. was added. All UCB monoclonal antibodies to surface antigens, including CD16 and CD56 (FITC; Serotec), were analyzed.

2.4 Result

Human umbilical cord blood (hUCB) and mononuclear cells (MNTCs) were treated with wheat protein extract (molecular weight>30 kDa, 100 μg/mL) for 7 days, the population of CD16⁺CD56⁺ NK-cells was 2.7 times higher than the population in the untreated control. This indicated that the wheat grass protein extracts (molecular weight>30 kDa) alter cell immunophenotypic expression in mononuclear cells (MNCs).

### Example 3

### Inhibition Effect on Nitrite Production by an Organic Solvent Extracts of Wheat Grass

3.1 Plant Materials

Plant materials were prepared as described in Example 1.1.

3.2 Preparation of Water Extract from Wheat Grass

150 g wheat grass powder was dissolved in 3,000 ml water and heated at 85° C. for 10 minutes. After centrifugation at 12,000 g for 15 minutes, the supernatant and pellet were respectively collected. The pellet was then dissolved in 2,000 ml of acetonitrile (ACN) for 6 hours at 25° C. After another centrifugation at 12,000 g for 15 minutes, the supernatant was concentrated into a pellet by a rotor vapor to yield 4.12 g of the acetonitrile extracts. Again the pellet was then dissolved in 2,000 ml of acetone for 6 hours at 25° C. After another centrifugation at 12,000 g for 15 minutes, the supernatant was concentrated into a pellet by a rotor vapor to yield 2.06 g of the acetone extracts. Finally, the pellet was then dissolved in 2,000 ml of hexane for 6 hours at 25° C. After another centrifugation at 12,000 g for 15 minutes, the supernatant was dried to yield 1.05 g of the hexane extracts.

3.3 Cell Lines and Cultures

RAW 264.7 cell line (ATCC TIB 71) was obtained from the American Type Culture Collection (Rockville, Md.). RAW 264.7 is a monocyte-macrophage cell line established from the ascites of a tumor induced in a male mouse by intraperitoneal injection of Abelson leukemia virus. These cells show pinocytotic and phagocytotic activities, secrete lysozyme and are capable of antibody-dependent lysis of both sheep erythrocytes and tumor targets. Cell lines were cultured routinely in Dulbecco's modified Eagle's medium (DMEM) supplemented with 10% fetal calf serum (FCS) (GibcoBRL), 100 U/ml of penicillin (Sigma), 0.1 U/ml streptomycin (Sigma) and 1% L-glutamine (Sigma). Cells were grown at 37° C. in an atmosphere of 5% CO₂ and 95% air.

3.4 Nitrite Release Assay

Since the NO radical quickly becomes NO₂ in aqueous solution, macrophage culture supernatants were assayed for NO₂ by a microplate assay method. Briefly, 100 μl of each supernatant was incubated in quadruplicates with an equal volume of Griess reagent (1% sulfanilamide, 0.1% N-(1-naphtyl)-ethylenediamide dihydrochloride, 2.5% H₂PO₄) for 10 minutes at room temperature. Absorbance at 570 nm was measured in a microplate reader. NO₂ concentration was standardized using NaNO₂.

3.5 Results

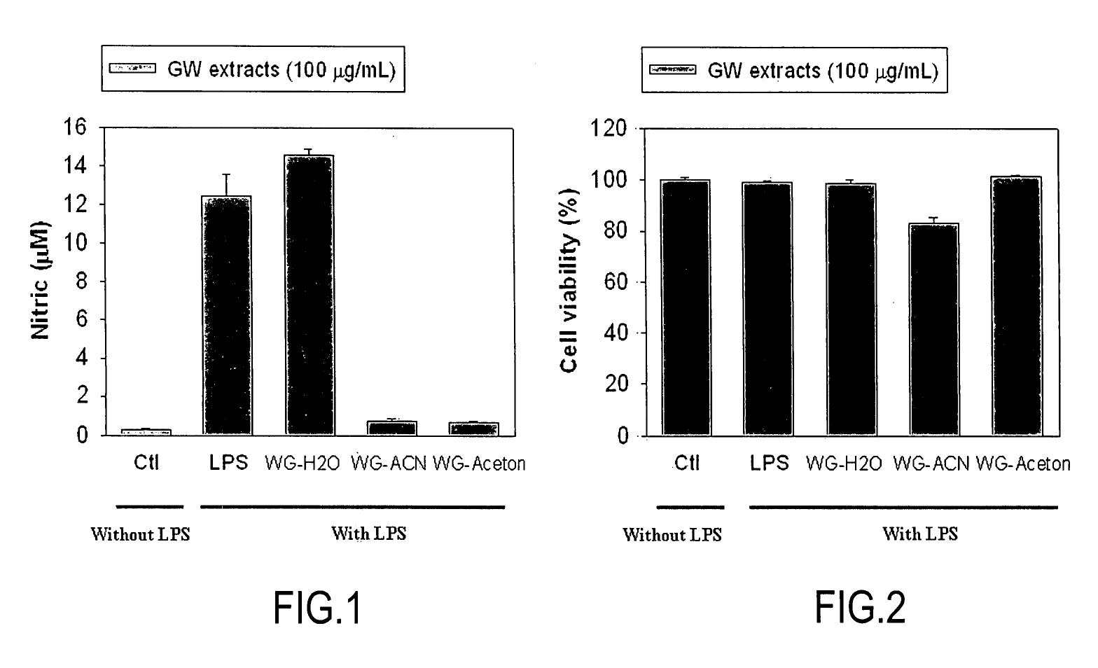

The wheat grass extracts extracted by ACN and acetone inhibited the nitrite production that was induced by lipopolysaccharides (LPS) in RAW 264.7 cells. However, with reference to FIGS. 1 and 2, the water-soluble wheat extracts did not inhibit the nitrite production after LPS treatment in RAW 264.7 cells.

### Example 4

### Anti-Oxidation Activity by Organic Solvent Extracts of Wheat Grass

4.1 Plant materials

Plant materials were prepared as described in Example 1.1.

4.2 Preparation of Wheat Organic Solvent Extracts

Wheat organic solvent extracts were obtained as described in Example 3.2.

4.3 Anti-Oxidation Bioactivity (1,1-dipheny-2-picrylhydrazyl (DPPH) Assay).

1,1-dipheny-2-picrylhydrazyl (DPPH) (16 mg) was dissolved in 100 ml of ethanol, 100 ml of distilled water was added, and the solution was filtered. Except where indicated, 500 μl of this DPPH solution was mixed with 50 μl of 0.1 M acetate buffer (pH 4.4) and 50 μl of wheat organic solvent extract, and made up to 1.0 ml with 18% ethanol. The solution was mixed and incubated at 50° C. The absorbance at 528 nm was measured after 20 min. A DPPH-scavenging ability unit (DU) was calculated as the difference between the absorbance of the reaction mixture at 528 nm with and without 50 μl of sake. Scavenging effect %= [symbol] 1−(Abs after/Abs before) [symbol] ×100%. Hydroquinone (HQ) was used as positive control. In the DPPH-scavenging assay, the more the DPPH free radicals are cleaned, the greater absorption value reduced.

4.4 Results

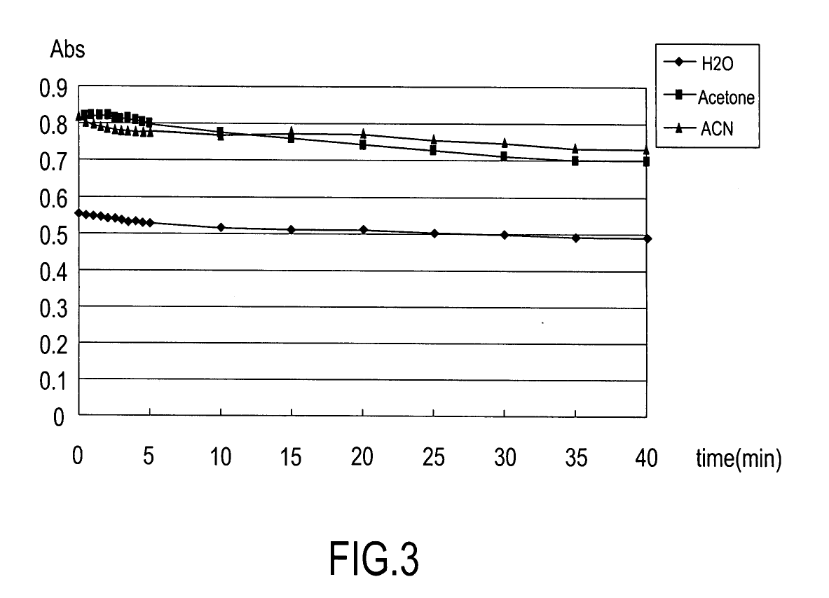

With reference to FIG. 3, the DPPH-scavenging activity of water is lower than the wheat grass extracts extracted by ACN and acetone. The scavenging effect of water is about 12%, the scavenging effect of the wheat grass extracts extracted by ACN is about 10%, and the scavenging effect of the wheat grass extracts extracted by acetone is about 13%. Therefore, the wheat grass extracts extracted respectively by ACN and acetone showed an excellent anti-oxidation activity by assaying the DPPH-scavenging activity.

### Example 5

### Regulation of the Uric Acid by Wheat Water-Soluble Extracts

Xanthine oxidase (XO) oxidizes oxypurines such as xanthine and hypoxanthine to uric acid. In humans, xanthine oxidase is normally found in the liver and not free in the blood. When the activity of xanthine oxidase is inhibited, the amount of uric acid would also be regulated.

5.1 Plant materials

Wheat grass (Triticum aestivum) was grown individually in 30 cm diameter×15 cm high containers. Water and fertilizer (Plantex 20-20-20, 500 mL of 0.6 g/L per pot per day) were applied by drip irrigation. The plants were grown in chambers with a 24° C. and 18° C. day-night temperature cycle and a 16 hour and 8 hour day-night photoperiod for 8 to 10 days. After harvesting, the wheat grass was milled in a laboratory-scale milling machine to obtain wheat grass juice. The wheat grass juice (2.2 L) was heated at 85° C. for 10 minutes. After centrifugation at 12,000 g for 15 minutes, the supernatant was freeze-dried to yield 98 g of the wheat water-soluble extracts. Samples were stored at −80° C. before use.

5.2 Active Components from Water Extract of Wheat Grass for Using Against Xanthine Oxidase

15 g of wheat water-soluble extract was re-dissolved in 100 ml of water and subjected to a preparative HPLC using a C18 packed column (10 mm×250 mm, 5 μm Spherical, Advanced Separation Technologies, Inc.) using stepwise elution with H₂O:AcCN:H₃PO₄ (87:13:0.01, solvent A) and H₂O:AcCN:H₃PO₄ (70:30:0.01, solvent B) to obtain active components. The elution started with solvent A and was conducted for 30 minutes at a flow rate of 12 ml/min, was changed to solvent B for 15 minutes, and then was changed back to solvent A.

The xanthine oxidase inhibitory activity was assayed spectrophotometrically at 295 nm under an aerobic condition. To perform an assay of xanthine oxidase inhibitory activity is well known in the art and suitable methods would be apparent to a skilled person in the art, so further description of the assay process is not provided.

5.3 Results

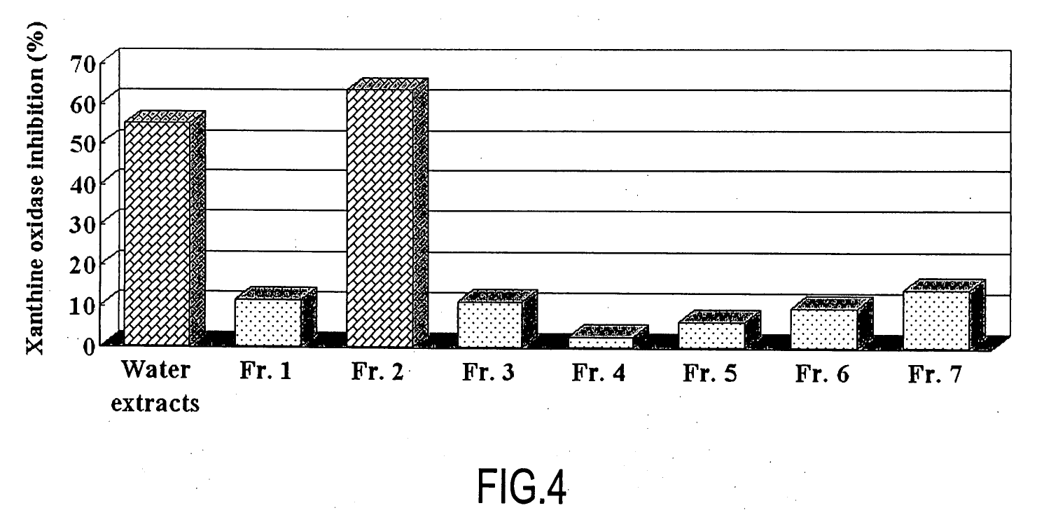

Wheat water-soluble extracts were first subjected to a preparative HPLC chromatogram. Seven fractions (Fr.) were obtained and collected respectively at the elution times of 0 to 4, 4 to 6.5, 6.5 to 10, 10 to 19, 19 to 23, 23 to 41 and 41 to 59 minutes. With reference to FIG. 4, among the fractions collected, its fractions were at a final concentration of 200 μl/ml. Each value is represented as mean±S.D. from triplicate measurements. The second fraction (elution time of 4 to 6.5) had the highest activity against xanthine oxidase. This fraction was then subjected to preparative HPLC chromatogram. Consequently, one active component was isolated with the molecular weight 136.0647. The results demonstrated that the active component is a pure compound that had activity against xanthine oxidase.

The active compound, 6-aminopurine isolated from wheat grass juice was analyzed using a high-resolution ESI-TOF mass spectrometer, and the molecular weight was 136.0647. This compound was then analyzed using NMR, and the results showed 2D ¹H-¹³C HMBC spectra were recorded with ²J or ³JH-C coupling constants at 8 and 5 Hz, 2D ¹H-¹³C HSQC spectra were recorded with ¹JH-C coupling constants at 145 Hz. ¹H NMR: δ 8.29 (s, 1H, H-8), δ 8.35 (s, 1H, H-2). ¹³C NMR: δ 115.8 (H-5), δ 142.3 (H-8), δ 148.4 (H-2), δ 149.7 (H-4), δ 152.4 (H-6). On the basis of the above data, this active compound was identified as 6-aminopurine by direct comparison with an authentic sample. The molecular formula of 6-aminopurine was C₅H₅N₅.

5.4 Assay of Xanthine Oxidase (XO) Inhibitory Activity of 6-aminopurine Analogues

6-aminopurine analogues were commercially obtained for assay of xanthine oxidase inhibitory activity.

The xanthine oxidase inhibitory activity was assayed spectrophotometrically at 295 nm under an aerobic condition. To perform the method for assaying the xanthine oxidase inhibitory activity by a spectrophotometer is well known in the art and suitable methods would be apparent to a skilled person in the art, so further description of the assay process is not provided. A reaction mixture containing 200 mM sodium pyrophosphate buffer (pH 7.5), 100 mM xanthine and 0.05 unit xanthine oxidase was prepared. The absorption increments at 295 nm indicating the formation of uric acid at 25° C. were followed, and the initial velocity was calculated. Wheat water-soluble extracts were dissolved directly in the buffer (200 mM sodium pyrophosphate buffer, pH 7.5), and wheat acetonitrile, acetone and hexane extracts obtained from Example 4.2 (please check) were dissolved initially in dimethylsulfoxide (DMSO) followed by dilutions with the buffer (200 mM sodium pyrophosphate buffer, pH 7.5) and was incorporated into the enzyme assay to assess the inhibitory activity. All determinations were performed in triplicate and samples were tested further to ascertain the corresponding IC₅₀ values.

5.5 Results of Xanthine Oxidase Inhibitory Activity of 6-aminopurine Analogues

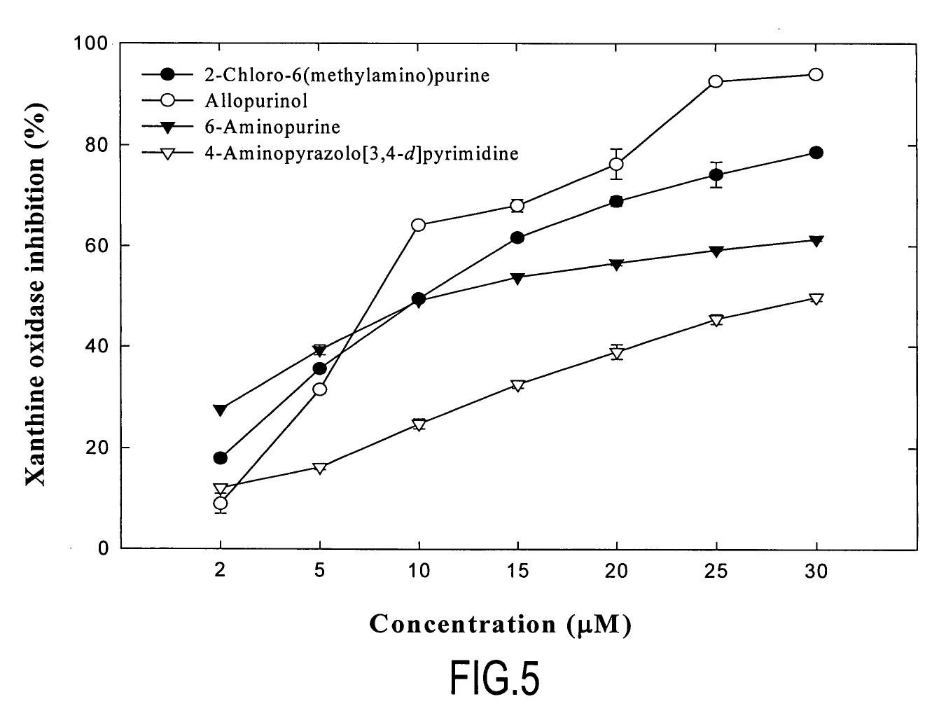

The name and structure of 6-aminopurine analogues are shown on Table 4, and the results of inhibitory effects of 6-aminopurine-based compounds including 6-aminopurine, 2-chloro-6(methylamino)purine and 4-aminopyrazolo[3,4-d]pyrimidine allopurinol on XOD activity are shown in FIG. 5. In FIG. 5, each point described indicates the average±S.D. of triplicate measurements. The inhibition effect of these compounds on the XO-catalyzed xanthine to uric acid reaction was determined. Allopurinol, 2-chloro-6(methylamino)purine, 6-aminopurine and 4-aminopyrazolo[3,4-d]pyrimidine showed a strong inhibitory effect on xanthine oxidase, and the IC₅₀ values of these compounds was 7.82±0.12, 10.19±0.10, 10.89±0.13 and 30.26±0.23, respectively. Moreover, 5-nitrobenzimidazole nitrate salt and 6-thioguanine showed an inhibitory effect on xanthine oxidase, and the IC₅₀ values of these compounds were 86.84±0.51 and 92.42±0.62, respectively. However, the others did not show a significant inhibitory effect on xanthine oxidase.

TABLE 3
Inhibition effect of 6-aminopurine analogues

| Serial | | | IC₅₀ ± SEM |
|---|---|---|---|
| number | Analogues | Structure | (μM) |
| 1 | Allopurinol | 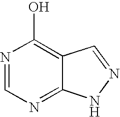 | 7.82 ± 0.12 |
| 2 | 2-Chloro-6(methylamino)purine | 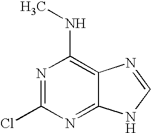 | 10.19 ± 0.10 |
| 3 | 6-Aminopurine | 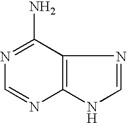 | 10.89 ± 0.13 |
| 4 | 4-Aminopyrazolo[3,4-d]pyrimidine | 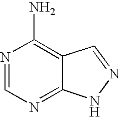 | 30.26 ± 0.23 |
| 5 | 5-Nitrobenzimidazole nitrate salt | 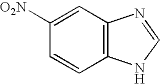 | 86.84 ± 0.51 |
| 6 | 6-Thioguanine | 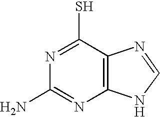 | 92.42 ± 0.62 |
| 7 | 2-Aminopurine | 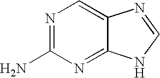 | >200 |
| 8 | 1,2,4-triazolo(1,5-a)pyrimidine | 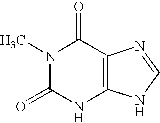 | >200 |
| 9 | 6-O-Methylguanine | 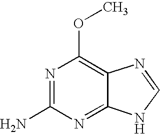 | >200 |
| 10 | 2-Amino-6-chloropurine | 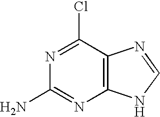 | >200 |
| 11 | 5-Methylbenzimidazole | 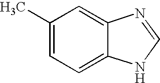 | >200 |
| 12 | 2,6-Diaminopurine | 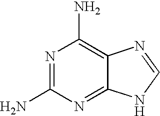 | >200 |
| 13 | 5,6-Dimethylbenzimidazole | 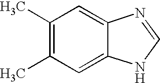 | >200 |

5.6 Mass Spectrometry

The 6-aminopurine analogues were further analyzed by mass spectrometry on a Finnigan LCQ Deca ion trap mass spectrometer (ThermoFinnigan, San Jose, Calif.) with an electrospray ionization interface. To conduct mass spectrometry experiments is well known in the art and suitable methods would be apparent to a skilled person in the art, so further description is not provided.

5.7 NMR Experiments

The 6-aminopurine analogues were further analyzed by NMR experiments. To conduct NMR experiments is well known in the art and suitable methods would be apparent to a skilled person in the art, so further description is not provided.

### Example 6

### Regulation of Blood Glucose by Wheat Water-Soluble Extracts

6.1 Plant Materials

Plant materials were prepared as described in Example 5.1.

6.2 Isolation of Active Components Having an Inhibiting Effect Against Xanthine Oxidase from Water Extract of Wheat Grass

15 g of water supernatant was further refrigerated and then was re-dissolved in 100 ml of water, and subjected to column chromatography on a C18 open column using elution with H₂O:AcCN (100:0) and H₂O:AcCN (0:100) to obtain active components.

6.3 Regulation of the Blood Glucose Level Assay (Insulin Assay)

HIT-T15 cells (10⁶/mL) were cultured in an F12K medium (2 mM L-glutamine, 1.5 g/L sodium bicarbonate, dialyzed horse serum (10%), FBS (2.5%) and 10 mM glucose) for 4 days, and then cultured in a Krebs medium for 1 hour. Wheat water-soluble extracts were then added to the medium for 1 hour, and the medium were collected for the insulin test. An insulin enzyme-linked immunosorbent (ELISA) kit (DSL Insulin ELISA (DSL-10-1600), Diagnostic Systems Laboratories) was used to assay the insulin activity. To operate the Insulin enzyme-linked immunosorbent (ELISA) kit is well known in the art and suitable methods would be apparent to a skilled person in the art, so further description is not provided.

6.4 Results of Regulation of the Blood Glucose Level Assay

The HIT-T15 cells treated with wheat water-soluble extracts showed a higher secretion of insulin. The insulin concentration of the treated HIT-T15 cells were 6.8˜9.8 μIU/mL, 100 μg/mL. The results are showed in Table 5.

TABLE 4
Secretion of insulin after treating HIT-T15 cells with wheat
water-soluble extracts

| | No. 1 | No. 2 | No. 3 | No. 4 | No. 5 | No. 6 | No. 7 | No. 8 | No. 9 |
|---|---|---|---|---|---|---|---|---|---|
| ΔAbs₄₅₀ | 0 | 0.005 | 0.027 | 0.062 | 0.071 | 0.089 | 0.008 | 0.004 | 0.010 |
| Insulin | 0 | 0.55 | 3 | 6.8 | 7.8 | 9.8 | 0.88 | 0.44 | 1.1 |
| conc. | | | | | | | | | |
| (μIU/ | | | | | | | | | |
| ml) | | | | | | | | | |

### Example 7

### Regulation of the Tyrosinase Activity by Wheat Water-Soluble Extracts

7.1 Plant Materials

Wheat grass (Triticum aestivum) was prepared as described in Example 2.1.

7.2 Isolation of Active Components Having an Inhibitory Effect Against Tyrosinase from Water Extract of Wheat Grass

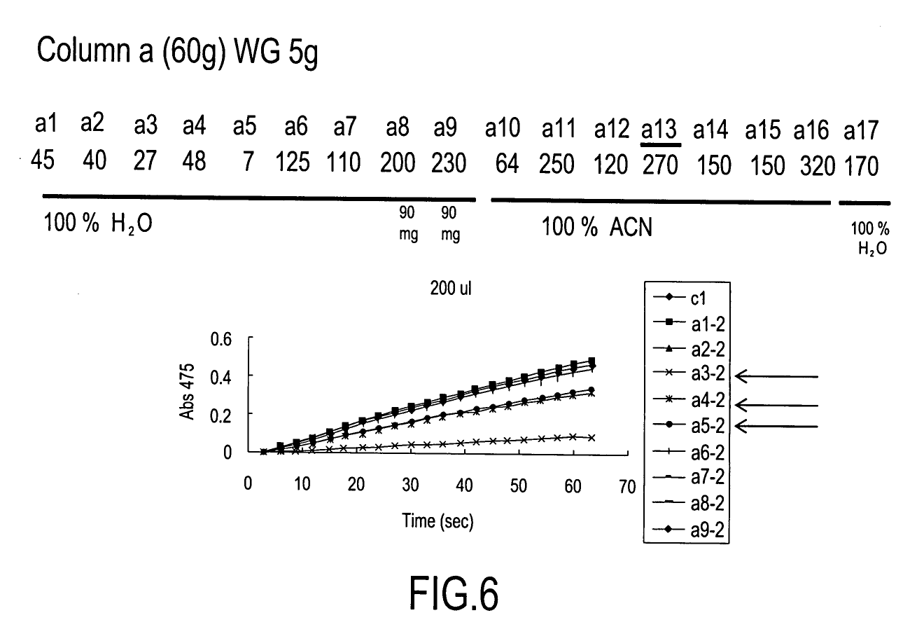

The method for isolating active compounds that have an inhibiting effect against tyrosinase from wheat water-soluble extract is the same as Example 6.2. Seven fractions (Fr.) were obtained and collected respectively at the elution times of 0 to 4, 4 to 6.5, 6.5 to 10, 10 to 19, 19 to 23, 23 to 41 and 41 to 59 minutes. Seven fractions were labeled as a1 to a17. FIG. 6 shows the fractions and the absorption value of each fraction during a certain time frame.

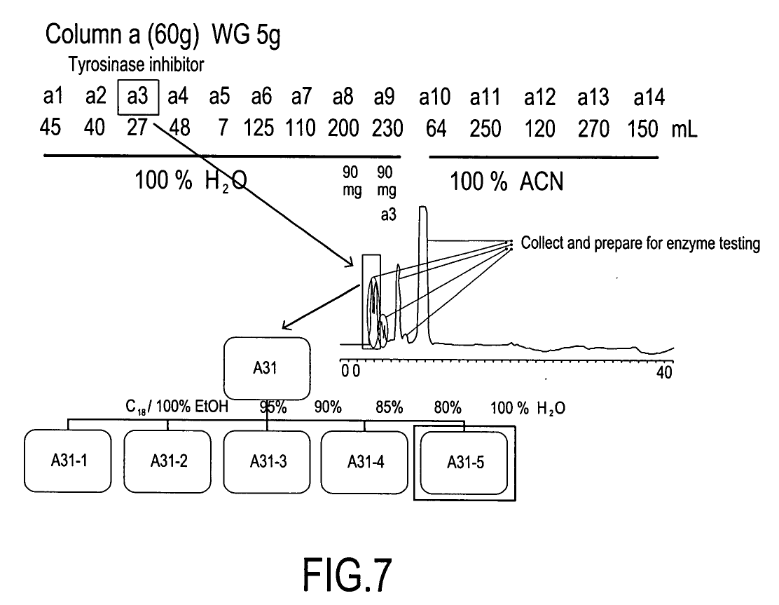

Wheat fractions labeled a3 were then subjected to column chromatography on a preparative HPLC using a C18 packed column (10 mm×250 mm, 5 μm Spherical, Advanced Separation Technologies, Inc.) was performed by elution with H₂O:AcCN:TFA (100:0:0.01) and H₂O:AcCN:TFA (0:100:0.01) to further isolate other active compounds. The fractions were labeled as a31. The fractions a31 were further purified, and the fractions were labeled as a31-1 to a31-5. FIG. 7 shows a scheme of further purification steps of wheat tyrosinase inhibitors.

The tyrosinase inhibitor was purified from the wheat water-soluble extracts by using a C18 open column and C18 packed column.

7.3 Assay of Tyrosinase Inhibitory Activity

With reference to FIG. 6, fractions labeled as a3-2, a-4-2 and a5-2 showed significant tyrosinase inhibitor activity. The inhibitory effect on tyrosinase was measured spectrophotometrically at 475 nm. A reaction mixture contained 370 μl of 50 mM sodium pyrophosphate buffer (pH 6.8), 200 μl of 2 mM L-DOPA, 100 μl of sample solution dissolved in distilled water or DMSO, 180 μl of distilled water and 150 μl of tyrosinase enzyme (300 unit/ml) was used to analyze the inhibitory effect. DMSO prevents the samples from dissolving in distilled water. The absorption increments at 475 nm indicated formation of uric acid at room temperature, and the initial velocity was calculated. The inhibitory activity of tyrosinase was assessed as % inhibition (1−β/α)×100, where α is the change in absorbance per minute without the sample (A blank with enzyme−A blank without enzyme), and β is the change in absorbance per minute with the sample (A test with enzyme−A test without enzyme).

7.4 Results of The Assay of Tyrosinase Inhibitory Activity

The a3, a31 and a31-5 were showed high activity. The IC₅₀ of the a3 fraction is 215 μg, the IC₅₀ of the a31 fraction is 121 μg, the IC₅₀ of the a315 fraction is 76 μg.

### Example 8

### Regulation of the Phosphodiesterases Activity by Wheat Water-Soluble Extracts

8.1 Plant Materials

Wheat grass (Triticum aestivum) was treated as described in Example 2.1.

8.2 Isolation of Active Components Having an Inhibitory Effect Against Phosphodiesterases from Wheat Water-Soluble Extracts

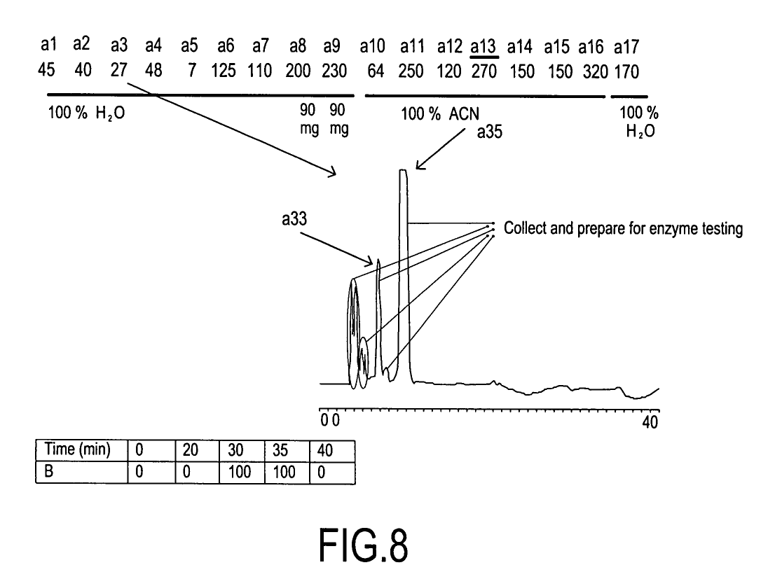

The method for isolating active components having an inhibitory effect against phosphodiesterases from wheat water-soluble extracts is the same as Example 7.2. FIG. 8 shows a graph of further purification of wheat phosphodiesterase inhibitor.

The phosphodiesterase inhibitor was purified from the wheat grass water extracts by using a C18 open column and C18 packed column. The phosphodiesterase inhibitor was purified from the a33 fraction. The phosphodiesterase inhibitor is trans-aconitic acid demonstrated by the MNR assay with formula of C₆H₆O₆, and the structure of C₆H₆O₆ is

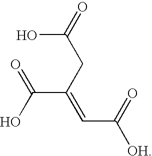

8.3 Regulation of the Phosphodiesterase Activity

The inhibitory effect on phosphodiesterases was measured spectrophotometrically at 405 nm. A reaction mixture containing 300 μl of 100 mM Tris buffer (pH 8.9), 600 μl of 1 mM Bis(p-nitrophenyl phosphate), 100 μl of sample and 100 μl of enzyme was used for analysis. The absorption increments at 405 nm indicated formation of uric acid at room temperature, and the initial velocity was calculated. The inhibitory activity of phosphodiesterases was assessed as % inhibition=(1−β/α)×100, where α is the change in absorbance per minute without the sample (A blank with enzyme−A blank without enzyme), and β is the change in absorbance per minute with the sample (A test with enzyme−A test without enzyme).

8.4. Results of the Assay of Phosphodiesterase Inhibitory Activity

Results indicated that the a3, a33 and a35 were showed high activity. The IC₅₀ of the a3 fraction is 215 μg, the IC₅₀ of the a33 fraction is 121 μg, the IC₅₀ of the a35 fraction is 76 μg.

### Example 9

### Immuno-Modulation Activity of Rice Protein Extracts

9.1 Plant Materials

Rice stem extracts (Oryza sativa L. Tainung 67) grown on a farm at a maximum daytime temperature of 32° C. and a minimum nighttime temperature of 25° C. After harvesting, the rice stems were milled with a laboratory-scale milling machine. Then the rice stem extract was filtered firstly through a 0.22 μm filter membrane, and then lyophilized and stored at −80° C. until use.

9.2 Isolation of Umbilical Cord Blood (UCB) Mononuclear Cells and the Method for Analysis

The method has described in Example 2.2.

9.3 Results of Rice Protein Extracts Treatment of UCB Mononuclear Cells

When human umbilical cord blood (hUCB) MTCs were treated with rice protein extracts (100 μg/mL) for 7 days, the populations of CD56⁺ NK cells, CD14⁺ monocyte/macrophage, CD83⁺ dendritic cells, CD3 T cell and CD19 B cell increased 3.8, 6.8, 4.2, 13.5 and 17.4%, respectively. This indicated that the rice protein extracts alter cell immunophenotypic expression in mononuclear cells (MNCs).

### Example 10

### Anti-Oxidation Activity by Organic Solvent Extracts of Rice Grass

SOD, an antioxidant enzyme, may be useful in the augmentation of antioxidant defenses in the endothelium. It also showed anti-aging bioactivity.

10.1 Plant Materials

Plant materials were prepared as described in Example 9.1.

10.2 Anti-Oxidation Bioactivity (Rice SOD Assay)

A BIOXYTECH SOD-525™ kit was used to assay the SOD activity. This kit contained a reagent R1 (5,6,6a,11b-tetrahydro-3,9,10-trihydroxybenzo [c]fluorene, in HCl containing diethylenetriaminepentaacetic acid (DTPA) and ethanol), a reagent R2 (1,4,6-trimethyl-2-vinylpyridinium trifluoromethanesulfo-nate, in HCl) and a buffer 2 (amino-2-methyl-1,3-propanediol, containing boric acid and DTPA, pH=8.8). Description of the assay procedure follows. First, the spectrophotometer was zeroed at 525±2 nm with deionized water. Second, 900 mL buffer 2 was added to a test tube for each blank or sample. Third, a 40 mL blank or sample was added to the test tube. Fourth, 30 mL of reagent R2 was added to the test tube and swirled. Fifth, samples with the above solutions were incubated at 37° C. for 1 minute. Sixth, 30 mL of reagent R1 was added to the test tube and vortex briefly. Seventh, samples were immediately transferred to a spectrophotometric cuvette and measured the absorbance over time.

Sample Calculation: 1). Rate Calculation: The autoxidation rate for the sample presented in the Rate Calculation section above was calculated by selecting the range of data from 0.4 to 0.7 minutes as the linear region of the curve. Then, a linear regression analysis was performed. The resulting slope of the line is 0.5452=Vs. Similarly, the average slope of four blanks was calculated as 0.0801=Vc. 2). Determine Vs/Vc Ratio:Vs/Vc=0.5452/0.0801=6.806. 3). Determine SOD Activity: (a) Using the Ratio Table, the corresponding activity is: Vs/Vc=6.80 is 9.35 units/mL. (b) Direct Calculation: {[0.93×(6.806−1)]/[1.073−(0.073×6.806)]}=9.372 units/mL.

10.3 Anti-Oxidation Bioactivity (DPPH Assay)

16 mg of 1,1-dipheny-2-picrylhydrazyl (DPPH) was dissolved in 100 ml of ethanol, than 100 ml of distilled water was added, and the solution was filtered. Except where indicated, 500 μl of this DPPH solution was mixed with 50 μl of 0.1 M acetate buffer (pH 4.4) and 50 μl of rice organic solvent extract, and made up to 1.0 ml with 18% ethanol. The solution was mixed and incubated at 50° C. The absorbance at 528 nm was measured after 20 minutes. A DPPH-scavenging ability unit (DU) was calculated as the difference between the absorbance of the reaction mixture at 528 nm with and without 50 μl of rice organic solvent extract. Scavenging effect %= [symbol] 1−(Abs after/Abs before) [symbol] ×100%.

10.4 Results of Anti-Oxidation Activity

Superoxide dismutase (SOD) activity of rice stem extracts were 64,135 units/Kg in Table 6. Furthermore, the water extracts of rice stem showed the DPPH-scavenging activity, and the EC₅₀ was 0.610 mg/ml.

TABLE 5
SOD activity of rice extracts

| | | | | Total activity |
|---|---|---|---|---|
| | | | SOD activity | (Units/kg |
| | Slop (/min) | Vs/Vc | (U/ml) | grass) |
| Rice juice | 0.225 | 5.92 | 178.5 | 64135 |
| Blank | 0.038 | | | |

### Example 11

### Regulation of the Uric Acid by Rice Water-Soluble Extracts

11.1 Plant Materials

Plant materials were prepared as described in Example 9.1.

11.2 Isolation of Active Compounds that Have an Inhibitory Effect Against Xanthine Oxidase from Rice Water-Soluble Extracts

Rice grass was prepared and assayed as described in Example 5.2.

11.3 Results

Rice water-soluble extracts were first subjected to a preparative HPLC chromatogram. One active compound was isolated and the molecular weight was 136.0647, namely 6-aminopurine. The results demonstrated that the pure compound could inhibit the activity of xanthine oxidase.

### Example 12

### Regulation of the Blood Glucose by Rice Water-Soluble Extracts

12.1 Plant Materials

Rice grass was prepared as described in Example 9.1.

12.2 Isolation of Active Components Having an Inhibitory Effect Against Xanthine Oxidase from Rice Water-Soluble Extracts and Regulation of Blood Glucose Level Assay (Insulin Assay)

Rice grass was prepared and assayed as described in Example 6.2 and 6.3.

12.3 Results

According to our results, the insulin concentration of HIT-T15 cells treated with 1 mg/mL from 15 day-old rice stem extracts was 1.53 μIU/mL.

### Example 13

### Regulation of the Tyrosinase Activity

13.1 Plant Materials

Plant materials were prepared as described in Example 9.1.

13.2 Isolation of Active Compounds that Have an Inhibitory Effect Against Tyrosinase

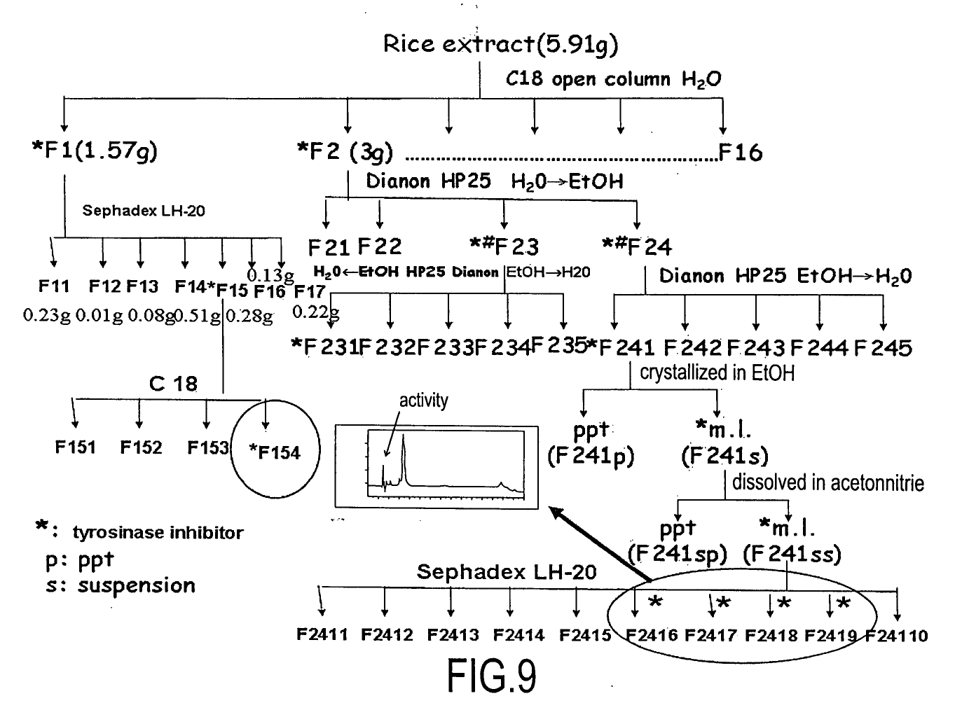

The method for isolating active compounds that have an inhibitory effect against tyrosinase was described in Example 6.2. FIG. 9 shows a scheme of purification steps of rice tyrosinase inhibitor.

13.3 Assay of Tyrosinase Inhibitory Activity

The method for assaying the tyrosinase inhibitory activity was described in Example 7.3.

13.4 Results

Two columns showed high tyrosinase inhibitory activity for the rice samples.

Various modifications and variations of the present invention will be recognized by people skilled in the art without departing from the scope and spirit of the invention. Although the invention has been described in connection with specific preferred embodiments, the invention as claimed should not be unduly limited to such specific embodiments. Indeed, various modifications of the described modes for carrying out the invention, which are obvious to those skilled in the art, are intended to be within the scope of the following claims.

1. A method for immuno-modulation in an organism, comprising administering to said organism an effective amount of a composition comprising a protein extract of a monocotyledon plant, wherein the protein extract is obtained by the steps of:
providing monocotyledon plant materials,
precipitating the monocotyledon plant materials with an ammonium sulfate solution, wherein the concentrations of ammonium sulfate are about 30% to about 100% (w/v), and
obtaining the protein extract which is precipitated from the ammonium sulfate solution.

2. The method as claimed in claim, wherein the protein extract is collected from 40% (w/v), 40% to 60% (w/v), 60% to 70% (w/v), 70% to 80% (w/v), or 80% to 100% (w/v) saturation of ammonium sulfate.

3. The method as claimed in claim 2, wherein the molecular weight of the protein extract is greater than 30 kDa.

4. A method for inhibiting nitrite production or anti-oxidation activity in an organism, comprising administering to said organism an effective amount of the composition comprising the acetonitrile extract, wherein the acetonitrile extract is obtained by the steps of:
dissolving the monocotyledon plant material in water and heated at about 85° C. for about 10 minutes,
(2) collecting water extract and a first pellet from the dissolving monocotyledon plant material, and
(3) treated the first pellet with acetonitrile for 6 hours to obtain an acetonitrile extract and a second pellet.

5. A method for regulating uric acid in an organism, comprising administering to said organism an effective amount of 6-aminopurine analogues.

6. The method as claimed in claim 5, wherein the 6-aminopurine analogues are allopurinol, 2-chloro-6(methylamino)purine, 6-aminopurine, 4-aminopyrazolo [3,4-d]pyrimidine, 5-nitrobenzimidazole nitrate salt or 6-thi9oguanine.

7. The method as claimed in claim 6, wherein the 6-aminopurine analogue is 2-chloro-6(methylamino)purine having formula I:

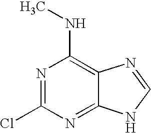

8. The method of claim 1, wherein said monocotyledon plant is a monocotyledon plant used in agriculture for the purpose of nourishment.

9. A method for immuno-modulation in an organism, comprising administering to said organism an effective amount of a composition comprising a protein extract of a monocotyledon plant,
wherein the monocotyledon plant is a wheat grass

10. The method according to claim 9, wherein the wheat grass is Triticum aestivum.

11. The method according to claim 9, where the protein extract is a precipitant of a wheat grass juice which is obtained by milling the wheat grass; and wherein the precipitant of the wheat grass juice is obtained by adding a percentage concentration of an ammonium sulfate to the wheat grass juice.

12. The method according to claim 11, wherein the protein extract is the precipitant of about 40% (w/v) of ammonium sulfate.

13. The method according to claim 11, wherein the protein extract is the precipitant of 40 to 60% (w/v) of ammonium sulfate.

14. The method according to claim 11, wherein the protein extract is the precipitant of 60 to 70% (w/v) of ammonium sulfate.

15. The method according to claim 11, wherein the protein extract is the precipitant of 70 to 80% (w/v) of ammonium sulfate.

15. The method according to claim 10, wherein the protein extract is the precipitant of 80 to 100% (w/v) of ammonium sulfate.

16. The method according to claim 10, wherein the molecular weight of the protein extract is greater than 30 kDa.
